(19) United States
(12) Patent Application Publication
(10) Pub. No.: US 2015/0230708 A1
(43) Pub. Date: Aug. 20, 2015

(54) METHODS AND SYSTEMS FOR DETERMINING VOLUMETRIC PROPERTIES OF A TISSUE

(71) Applicant: UNIVERSITY OF WASHINGTON THROUGH ITS CENTER FOR COMMERCIALIZATION, Seattle, WA (US)

(72) Inventors: Ruikang K. Wang, Seattle, WA (US); Murray Johnstone, Bainbridge Island, WA (US)

(21) Appl. No.: 14/421,926

(22) PCT Filed: Aug. 23, 2013
(86) PCT No.: PCT/US2013/056395

§ 371 Date: Feb. 16, 2015

Related U.S. Application Data
(60) Provisional application No. 61/692,638, filed on Aug. 23, 2012.

(51) Int. Cl. A61B 5/00; A61B 3/10; A61B 3/12

(52) U.S. Cl. CPC A61B 5/0066; A61B 3/1233; A61B 3/102; A61B 5/7278

(57) ABSTRACT

Systems and methods for determining microvascular functions in a sample of a subject are provided. A system obtains one or more spectral interference signals from the sample during one or more scans, extracts data from the spectral interference signals concerning cell, tissue, or particle motion within the sample via one or more optical microangiography algorithms, and calculates volumetric properties from the data indicative of fluid motion within the sample. The system and method may be used for diagnosing, providing a prognosis, or monitoring treatment of a disorder of the sample.

### CROSS-REFERENCE TO RELATED APPLICATIONS

This application claims priority to U.S. Provisional Patent Application Ser. No. 61/692,638 filed on Aug. 23, 2012, which is hereby incorporated by reference in its entirety.

### BACKGROUND

The assessment of both the structure of a living tissue and microvascular functions in the living tissue provides important information for diagnostics, treatment, and/or management of pathological conditions.

Ocular perfusion within the retina and choroid of the eye may be assessed to diagnose, treat, and monitor a number of pathological conditions in ophthalmology, such as glaucoma, papilledema, idiopathic and inflammatory forms of optic neuritis, and ischemic optic neuropathies. Such assessments may be used to provide guidance in medical, laser, or surgical management for a disorder of the tissue of the eye. Disorders of a tissue of the skin, heart, vasculature microcirculation, connective tissue structures, internal organs, and central nervous system are other examples of conditions where measuring microvascular properties may be beneficial.

Techniques have been developed to visualize and measure in vivo blood flow. However, such techniques either only provide information on flow in a single vessel without providing information about perfusion of the remaining area of interest, or are limited to a small selected area and the exact volume of the tissue being measured is not known, restricting the reproducibility of measurements. Traditional methods have also been insufficient to measure the depth-resolved structure and blood flow within the microcirculation of tissue non-destructively in vivo.

There is a need for a sensitive, non-invasive method and system for assessing properties related to the blood flow living tissue of a subject.

### SUMMARY

In accordance with the present invention, a system and a method are defined for determining microvascular functions in a sample of a subject. In one embodiment, the method may comprise performing a repeated scan of the sample with a probe beam from a light source, obtaining one or more spectral interference signals from the sample during the scan, extracting data from the spectral interference signals concerning cell, tissue, or particle motion within the sample, and calculating volumetric properties from the data indicative of fluid motion within the sample. The data from the spectral interference signals concerning cell, tissue, or particle motion within the sample may be extracted via one or more optical microangiography algorithms. The method may be used for diagnosing, providing a prognosis, or monitoring treatment a disorder of a sample, such as a living tissue in a subject, for example. Particularly, the subject may be at risk of an ocular pathology or has an ocular pathology. The ocular pathology may be but is not limited to one or more of glaucoma, papilledema, inflammatory neuropathies, and ischemic neuropathies.

In one embodiment, the method may further comprise combining a UHS-OMAG imaging protocol with a D-OMAG imaging protocol, wherein the D-OMAG imaging protocol comprises performing the repeated scan of two or more scans at a location, followed by using the phase difference between adjacent A-scans to extract volumetric properties, and the UHS-OMAG protocol comprises performing a plurality of fast scans on a fast scan axis with the probe beam from the light source, performing a plurality of slow scans on a slow scan axis, obtaining a data set from the plurality of fast and slow scans, producing at least one microstructural image of the sample, and mapping the determined volumetric properties into the microstructural image of the tissue. An imaging algorithm may be applied to produce at least one microstructural image of the sample. In an alternative embodiment, the method may further comprise combining a UHS-OMAG imaging protocol with OMAG imaging protocol.

In another embodiment, a system for measuring microcirculation is provided.

The system includes an optical coherence tomography probe, an optical circulator, a coupler, a spectrometer, and a physical computer-readable storage medium. The system is configured to acquire images from living tissue. The physical computer-readable storage medium has stored thereon instructions executable by a device to cause the device to perform functions to extract microcirculation data from images acquired from optical coherence tomography scans of the tissue, the functions comprising: determining a phase difference and a time interval between adjacent A-lines from the acquired images, calculating an axial velocity for the at least one vessel from the determined phase difference and the time interval, determining a Doppler angle and a diameter of at least one vessel from the acquired images, and calculating blood flow velocity from the axial velocity, the Doppler angle, and the diameter of the at least one vessel.

These as well as other aspects and advantages of the synergy achieved by combining the various aspects of this technology, that while not previously disclosed, will become apparent to those of ordinary skill in the art by reading the following detailed description, with reference where appropriate to the accompanying drawings.

### BRIEF DESCRIPTION OF THE FIGURES

FIG. 1 depicts a block diagram of an imaging apparatus in accordance with at least one embodiment;

FIG. 2 depicts a schematic of an exemplary system in accordance with at least one embodiment;

FIG. 3 depicts an image of an ONH taken with the exemplary system of FIG. 2 in accordance with at least one embodiment;

FIG. 4a depicts a fundus image of the temporal region of the ONH taken with the exemplary system of FIG. 2 in accordance with at least one embodiment;

FIG. 4b depicts the projection image of corresponding 3D microvasculatures for the image of FIG. 4a in accordance with at least one embodiment;

FIG. 4c depicts an example cross-sectional image generated by the exemplary system of FIG. 2 at the position marked as the dashed line in FIG. 4a, in accordance with at least one embodiment;

FIG. 4d depicts a corresponding blood flow image for FIG. 4c, in accordance with at least one embodiment;

FIG. 5 depicts a volumetric rendering for a 3D dataset, in accordance with at least one embodiment;

FIGS. 6a-l depict images of enface slices taken from the superficial surface of the nerve layer of a living tissue, in accordance with at least one embodiment;

FIG. 7a depicts an image of an enface microstructure image, in accordance with at least one embodiment;

FIG. 7b depicts a binary image of the image of FIG. 7a, in accordance with at least one embodiment;

FIG. 7c depicts an image resulting from superimposing the image in FIG. 7b over the image in FIG. 7a, in accordance with at least one embodiment;

FIG. 8a depicts a UHS-OMAG microangiogram of choroidal and ONH capillary beds at increasing IOP, in accordance with at least one embodiment;

FIG. 8b depicts a graph illustrating the effect of IOP on RBF as a percent of baseline plotted over mmHg, in accordance with at least one embodiment;

FIG. 8c depicts a graph illustrating the vessel diameter change as a percent of baseline plotted over mmHg, in accordance with at least one embodiment;

FIG. 9a depicts a UHS-OMAG microangiogram of choroidal and ONH capillary beds after removal of the retinal vessels, in accordance with at least one embodiment;

FIG. 9b depicts a graph illustrating the effect of IOP on choroidal perfusion as a percent of baseline plotted over mmHg, in accordance with at least one embodiment;

FIGS. 10a-d depict OCT structural images and corresponding flow images, in accordance with at least one embodiment;

FIGS. 10e-h depict steps in quantitation of ONH blood perfusion, in accordance with at least one embodiment; and

FIG. 11 depicts the effect of IOP on ONH blood perfusion as a signal volume percent plotted over mmHg, in accordance with at least one embodiment.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Embodiments described herein provide an ultrahigh sensitive optical microangiography (UHS-OMAG) system that delivers high sensitivity with a relatively low data acquisition time. OMAG is an imaging modality that is a variation on optical coherence tomography (OCT). The imaging is based on the optical signals scattered by moving particles. The light backscattered from a moving particle may carry a beating frequency that may be used to distinguish scattering signals by the moving elements from those by the static elements. Thus, the optical signals backscattered from the moving blood cells are isolated from those originated from the tissue microstructures. Accordingly, OMAG can be used to image the flow of particles, such as blood flow. As an extension to the OMAG imaging technique, Doppler OMAG (D-OMAG) uses the phase difference between adjacent A-scans of OMAG flow signals to extract volumetric properties, such as the flow velocity.

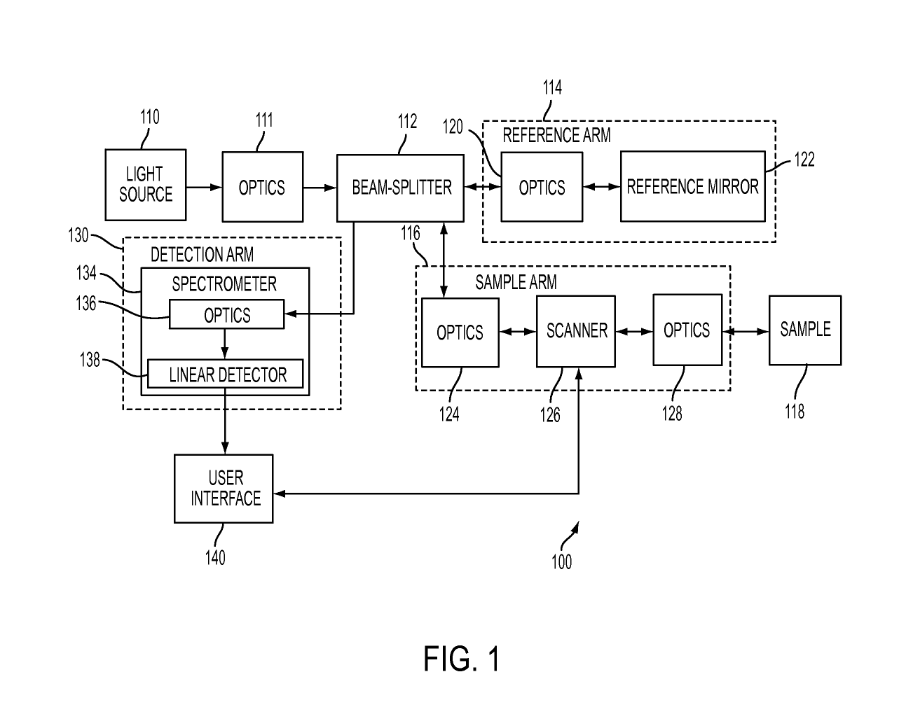

FIG. 1 depicts a block diagram of an imaging apparatus in accordance with at least one embodiment. The imaging apparatus may be a UHS-OMAG apparatus 100 suitable for ultrahigh sensitive 2-D and 3-D flow imaging. The illustrated UHS-OMAG apparatus 100 may include some features known in the art, features which may not be explained in great length herein except where helpful in the understanding of embodiments of present disclosure.

The UHS-OMAG apparatus 100 may be used, among other things, to measure biomechanical properties of a living tissue sample of a subject. Thus, the UHS-OMAG apparatus 100 may be used on a subject in vivo. As referenced herein, a subject may be a human subject.

As shown in FIG. 1, UHS-OMAG apparatus 100 includes a light source 110. In one example embodiment, light source 110 comprises a broadband light source, for example, superluminescent diode with a central wavelength of 1310 nanometers (nm) and a full-width-at-half-maximum bandwidth of 65 nm. In some example embodiments, light source 110 comprises a light source haying one or more longer or shorter wavelengths, which may allow for imaging at deeper levels in a sample. In other example embodiments, light source 110 may comprise a tunable laser source, such as, for example, a swept laser source.

UHS-OMAG apparatus 100 may include optics 111 to couple she light from the light source 110 into the system. The UHS-OMAG apparatus 100 may include a beam splitter 112 for splitting the light from optics 111 into two beams: a first beam provided to a reference arm 114 and a second beam provided to a sample arm 116. In some example embodiments, optics 111 may include various lenses or fiber optics components suitable for use with the apparatus 100. Beam splitter 112 may comprise a 2×2 single-mode fiber coupler, in one example embodiment.

Reference arm 114 may be configured to provide a reference light to a detection arm 130, from the light provided by light source 110, for producing a spectral interferogram in combination with backscattered light from a sample 118. Reference arm 114 may include optics 120 and a reference minor 122 for reflecting light from light source 110 for providing the reference light. Optics 111 may include various lenses suitable for use with the apparatus 100.

Reference mirror 122 may be stationary or may be modulated. Modulation may be equivalent to frequency modulation of the detected signal at detection arm 130. Spectral interference signals (interferograms) play be modulated by a constant Doppler frequency by a modulated mirror in the reference arm 114. The spectral interference signal may then be recovered by de-modulating the modulated signal at the modulation frequency. De-modulation may be achieved using any suitable method including, for example, a digital or optical de-modulation method. Modulation and de-modulation of spectral intereference signals may advantageously improve the signal-to-noise ratio, resulting in an improved image quality for the structural, flow, and angiographic imaging.

Sample arm 116 may be configured to provide light from light source 110 to a sample 118 by way of optics 124, a scanner 126, and optics 128. Optics 124 may be used to couple the light from beam splitter 112 to scanner 126. Optics 128 may include various optical lenses, for example, an optical collimator. Scanner 126 may include a pair of x-y galvanometer scanners for scanning sample 118 in an x-y direction. Optics 111 may comprise the appropriate optics for delivering the light form the scanner 126 onto sample 118. In some example embodiments, scanner 126 may also receive backscattered light from sample 118.

The light returning from reference arm 114 and from sample arm 116 may be recombined and coupled into the beam splitter 112 for introduction to the detection arm 130. As shown in FIG. 1, detection arm 130 comprises a spectrometer 134 including one or more of various optics 136 including, one or more collimators, one or more diffracting/transmission gratings, and one or more lenses (not illustrated). In an exemplary embodiment, optics 136 includes a 30-millimeter (mm) focal length collimator, a 1200 lines/mm diffracting grating, and an achromatic focusing lens with a 150 mm focal length.

In embodiments employing a broadband light source, spectrometer 134 may include a detector, such as a linear detector 138, configured to detect a spectral intereference signal. Linear detector 138 may include one or more of a line-scan camera and an area scan camera. One example linear detector 138 is a charge-coupled device (CCD). Other linear detectors may also be envisioned.

In embodiments where light source 110 comprises a tunable laser rather than a broadband light source, however, UHS-OMAG apparatus 100 may include a diffusion amplifier that may comprise one or more single element detectors rather than spectrometer 134. For example, one or more dual-balanced photo-diode detectors may be used.

In sonic example embodiments, UHS-OMAG apparatus 100 may include one or more user interfaces 140 for one or more purposes including controlling linear detector 138 and scanner 126, computing data using algorithms, displaying images, input of data, output of data, and the like.

UHS-OMAG apparatus 100 may be configured to build a 3D data volume set by scanning sample 118 with a sample light in x, y, and λ (z) directions to obtain a 3D spectral interferogram data set. Such a 3D data volume set may be built using methods described in U.S. patent application Ser. No. 13/577,857, entitled “Method and Apparatus for Ultrahigh Sensitive Optical Microangiography,” which is incorporated herein by reference.

In some example embodiments, scanner 126 may include an x-scanner and a y-scanner. During the composite scan, the x-scanner may perform at least one fast scan along a fast scan axis, and the y-scanner may perform at least one slow scan along a slow scan axis. The fast scan axis may be orthogonal to the slow scan axis. The fast scan may also be referred to as the axis, the lateral axis, and/or the B-scan axis. Similarly, the slow scan may also be referred to herein as a C-scan, and the slow scan may also be referred to as the y-axis, the elevational axis, and/or the C-scan axis. Each fast scan may be performed over a fast scan time interval, and each slow scan may be performed over a slow scan time interval, where the slow scan time interval is at least twice as long as the fast scan time interval. In some embodiments, the scanner may perform the one or more fast scans contemporaneously with the one or more slow scans. In such embodiments, a plurality of fast scans may be performed during one slow scan. A combination of slow and fast scans provides a 3D data set necessary to obtain a 3D image. Thus, a UHS-OMAG imaging protocol comprises a plurality of fast scans on the fast scan axis and a plurality of slow scans on the slow scan axis.

In each B-scan there may be a number of A-scans. An A-scan may be performed in the z-axis, orthogonal to both the x-axis and the y-axis. Each A-scan may include a number of pixels, i.e., data points, providing imaging depth information in the z-axis. Similarly, a C-scan may include a number of B-scans.

In some example embodiments, an imaging algorithm may be applied to the 3D data set to produce at least one image. The imaging algorithm may be applied on the slow scan axis. In some example embodiments, the imaging algorithm may separate a moving component from a structural component of the sample. The image may be a full range structural image, and/or a separated structural flow image, in some example embodiments, the image may be of blood flow, such as blood flow in the eye.

### Example 1

### Imaging and Assessment of a Human Eye In Vivo

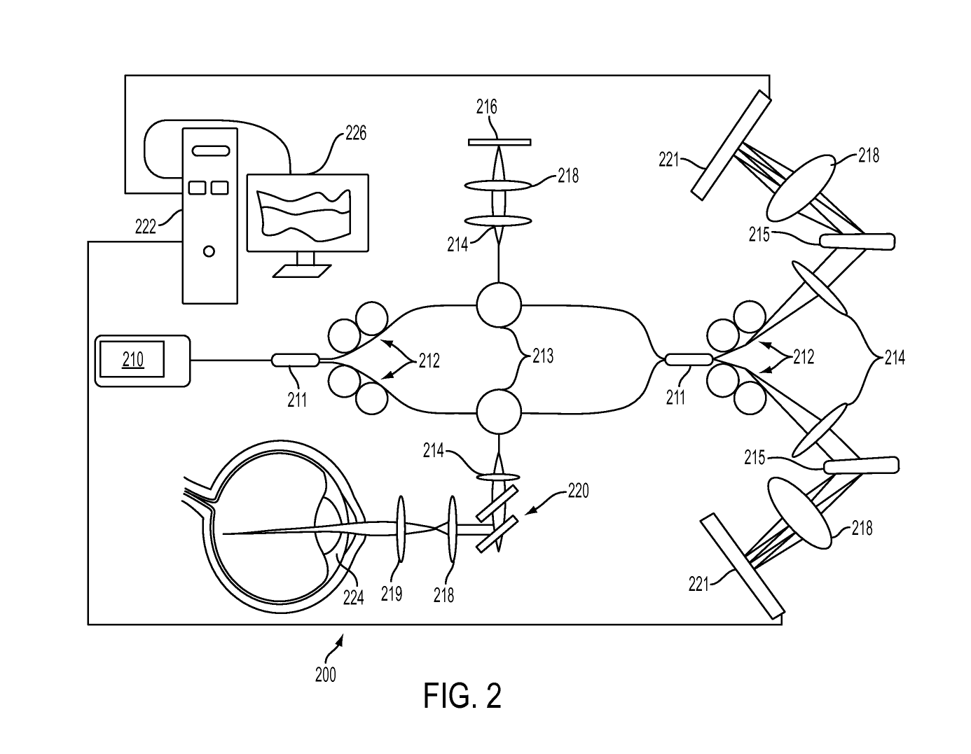

FIG. 2 depicts a schematic of an exemplary system 200 that was used to image and assess a human eye in vivo. The exemplary system in FIG. 2 is an OMAG system 200 comprising a light source 210, a fiber optic coupler 211, polarization controllers 212, optical circulator 213, collimators 214, diffraction gratings 215, a reference mirror 216, focusing lenses 218, an ocular lens 219, an X-Y galvanometer 220, line scan cameras 221, a main computing system 222, and a display 226. A sample 224 is positioned to be imaged and assessed. The system 200 may be similar to a described in L. An, P. Li, T. T. Shen and K. Wang, High Speed Spectral Domain Optical Coherence Tomography for Retinal imaging at 500,000 A-lines per Second, Biomed Opt. Express 2(1), 2770-2783 (2011).

Example 1 is discussed in detail in An, L. Johnstone, and Wang, R. Optical Microangiography Provides Correlation Between Microstructure and Microvasculature of Optic Nerve Head in Human Subjects, Journal Biomedical Optics 17:116018-116018, 2012. In Example 1, the light source 210 comprised a superluminescent diode with a spectral bandwidth of 45 nm centered at 842 nm that provided an axial resolution in air of about 7 μm. The light source 210 was coupled to a fiber-based Mach-Zehnder interferometer via a 20/80 fiber coupler. In Example 1, using two optical circulators, 20% of the light was routed to the sample arm and 80% to the reference arm. In the sample arm, the light was delivered into the sample 224 via a scanning optics setup with a measured light power of about 0.8 mW at the cornea. The scanning optics comprised collimators 214, X-Y galvanometer 220, and ocular lens 219, which provided a raster-scanning of the probe-beam spot at the retina.

The main computing system 222 used in Example 1 may be the same as or similar to any number of computing systems known in the art and may include a processor, data storage, and logic. These elements may be coupled by a system or bus or other mechanism. The processor may include one or more general-purpose processors and/or dedicated processors, and may be configured to perform an analysis on the output generated from the line scan cameras in the system 200. An output interface may be configured to transmit output from the computing system to a display, such as the display 226.

The light backscattered from the eye and reflected from the reference mirror 216 was collected and delivered to two high-speed spectrometers via fiber coupler 211. For each line scan camera 221, 800 out of 4096 pixels were selected for sensing the spectral interferogram, resulting in a 250 kHz A-scan rate.

A system such as the system 200 used for Example 1 may be used to image and assess tissue organization and microvascular functions within the optic nerve head (ONH), in one example embodiment, in a non-invasive and concurrent manner. The ONH comprises a superficial nerve layer, pre-lamina, lamina cribrosa, and retro-lamina regions.

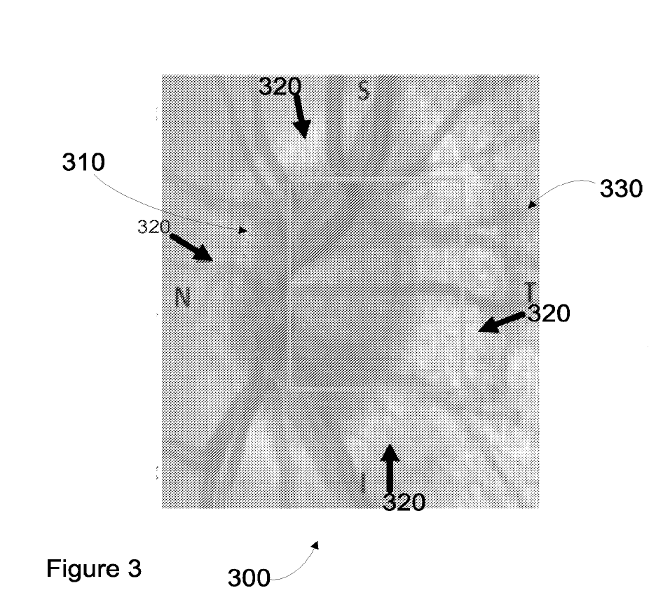

FIG. 3 depicts an image of an ONH 300 taken with the system of FIG. 2. The image 300 is a volumetric Fourier domain optical coherence tomography (FDOCT) image covering an area of about 3×3 mm² centered on the ONH by use of a low lateral resolution imaging probe. To obtain the image 300, 500 A-scans and 500 B-scans were taken and the A-scans were integrated along the z-axis direction in the ONH.

The image 300 shows features including the optic disk represented by dashed line circle 310, scleral rim identified as a region by arrows 320, and the ONH blood vessels, such as a blood vessel 330. Furthermore, S denotes superior, N denotes nasal, T denotes temporal, and I denotes inferior. However, this image does not show the detailed microstructural and microvascular morphology of the ONH.

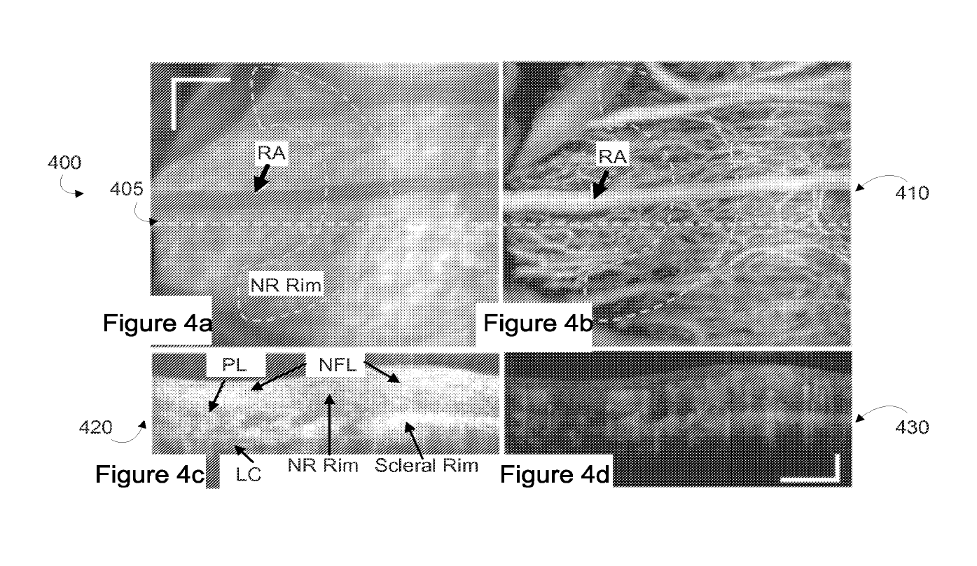

FIG. 4a depicts a fundus image 400 of the temporal region of the ONH taken with the system of FIG. 2. To obtain the image 400, a high resolution optical imaging probe with a probe beam diameter of approximately 4 mm at the cornea was installed on the sample arm to deliver a lateral resolution of about 6 μm at the ONH. To obtain imaging of the ONH microcirculation, 500 pixels were captured along the fast-scan direction and 1500 B-frames along the slow-scan direction. In image 400, RA depicts the retinal artery, and NR rim the neuroretinal rim.

FIG. 4b depicts the projection image 410 of corresponding 3D microvasculatures for the image of FIG. 4a in accordance with at least one embodiment. FIG. 4b shows that the ONH is highly vascularized, evidence until now which was only obtainable through in vitro corrosion casting techniques or histologic study. In this Example 1, the blood flow in the vessels within the ONH can be localized to an ONH depth of about 0.8 mm.

FIG. 4c depicts an example cross-sectional image 420 generated by the system 200 of FIG. 2 at the position marked as a dashed line 405 in FIG. 4a, and FIG. 4d depicts a corresponding blood flow image 430 for FIG. 4c. In FIG. 4c, NFL represents the nerve fiber layer, PL the prelaminar ONH tissue, and LC the lamina cribrosa.

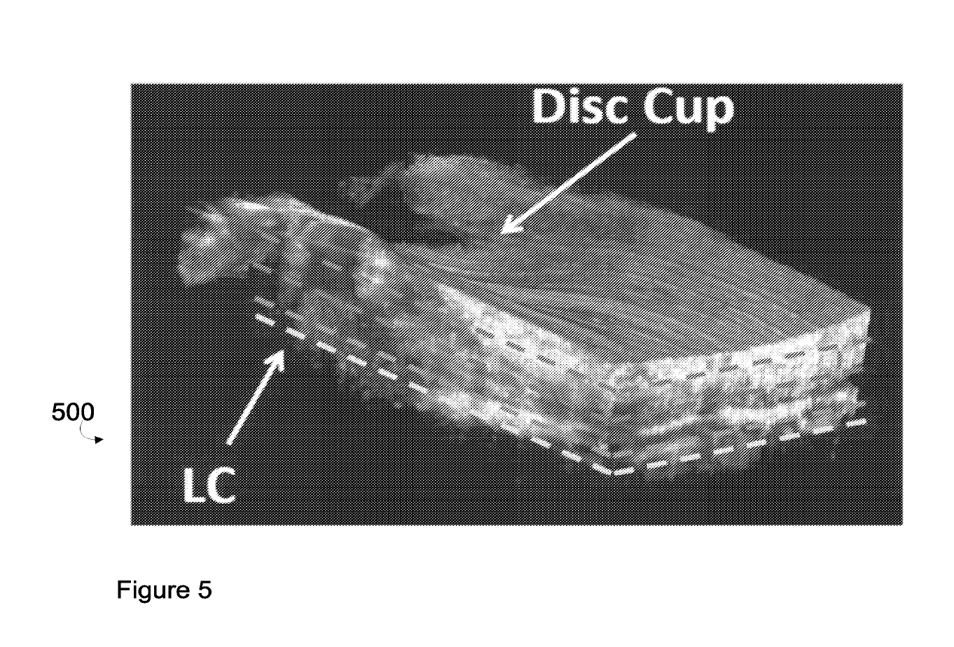

FIG. 5 depicts a volumetric rendering 500 for a 3D dataset obtained using the system 200 of FIG. 2. The volumetric rendering 500 shows locations of some key features of the ONH such as the lamina cribrosa (represented by LC) and cupped region “Disc Cup” of the optic nerve. The 3D dataset comprises the structural, organizational, and vascular information and can be manipulated to display enface tissue slices at particular depths.

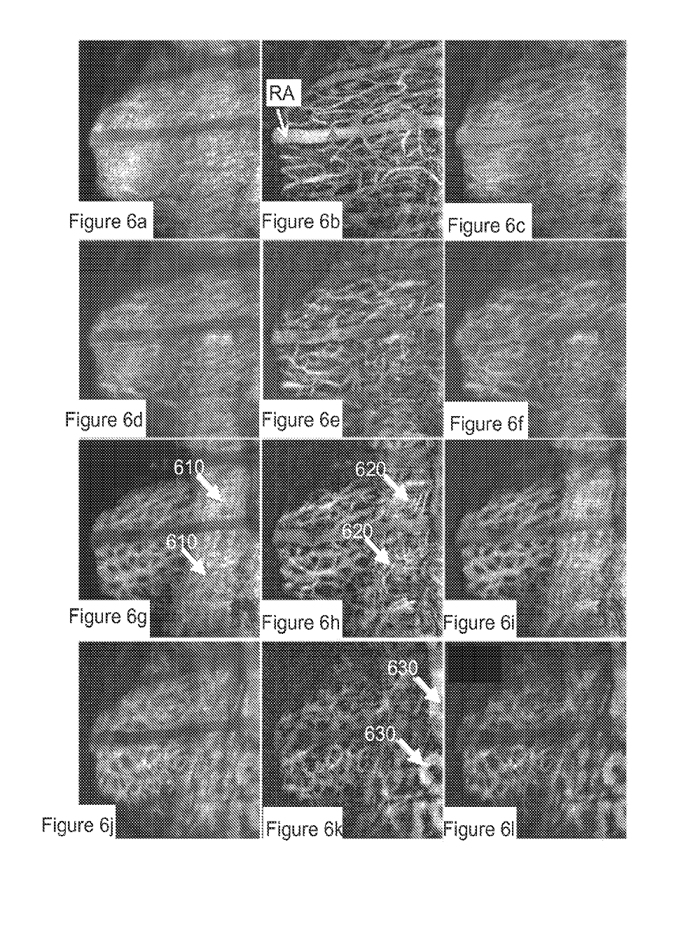

One such example of enface tissue slices is shown in FIGS. 6a-l, which depict images of enface slices taken from the superficial surface of the nerve layer of a living tissue. FIGS. 6a,d, g, and j illustrate enface tissue slices of the microstructural images, FIGS. 6b, e, h, and k illustrate corresponding vascular images, and FIGS. 6c, f, i, and l illustrate merged structure and vascular images, allowing for a better appreciation of their spatial relationships. The images of FIGS. 6a-c were extracted at about 80 μm (to correspond with the superficial nerve fiber layer), the images of FIGS. 6d-f at about 180 μm (to correspond with the pre-lamina), the images of FIGS. 6g-i at about 400 μm (to correspond with the lamina cibrosa), and the images of FIGS. 6j-l at about 600 μm (to correspond with the lamina cibrosa).

The high resolution and sensitivity of the system 200 permitted visualization of the scleral rim (identified by the arrows 610 in FIG. 6g) underlying the nerve fiber bundles entering the ONH around the ONH circumference. The scleral rim tissue is highly vascularized (shown by arrows 620 in FIG. 6h), a property not easily appreciated when examining histologic material in which the circulation is devoid of blood. Additionally, Example 1 demonstrates the ability of OMAG system 200 to visualize the larger blood vessels lying within the scleral rim periphery adjacent to the deeper region of the lamina cribrosa (shown by arrows 630 in FIG. 6k).

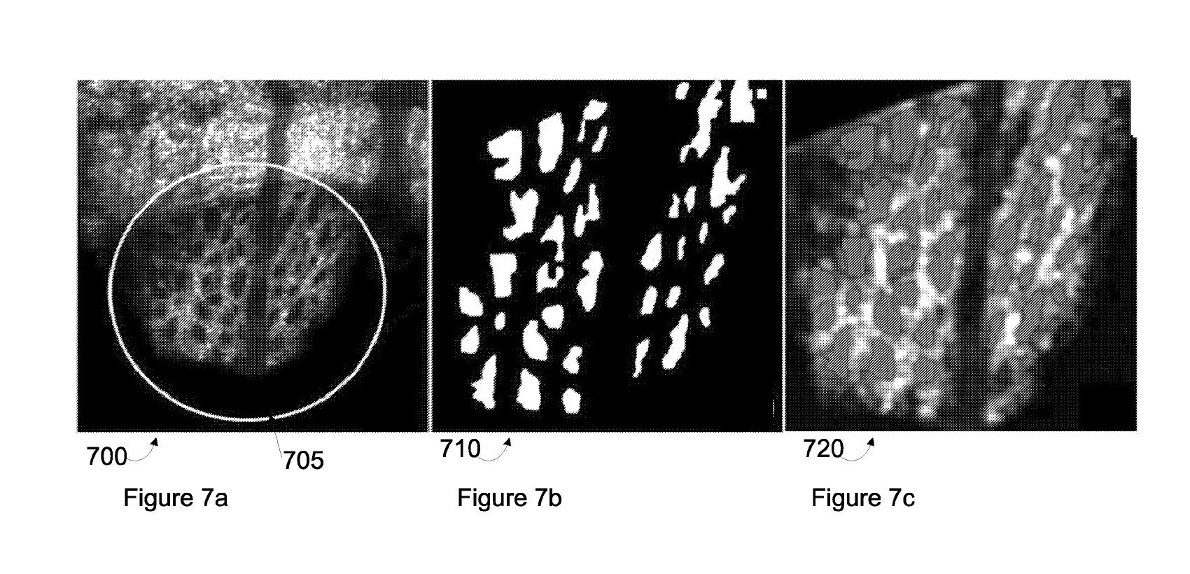

The total intravascular volume within the 3D tissue volume may be quantified. For example, FIG. 7a depicts an image 700 of an enface microstructure image obtained with the results obtained from the sample 224 examined using system 200. The porous structure of the lamina cribrosa can be easily viewed in FIG. 7a, which was taken at a depth of about 400 μm below the ONH surface. To quantitatively evaluate the distribution of the pore sizes, the LC region in FIG. 7a is manually selected and marked with the white circle 705. The pore areas were then isolated, as depicted in the binary image 710 of FIG. 7b by use of a binarization method. FIG. 7c depicts an image 720 resulting from superimposing the image in FIG. 7b over the image in FIG. 7a. The pore area and elongation ratio (ratio of major to minor axes of an ellipse that fits the pore) can be evaluated using a method such as that in K. M. Ivers et al., Reproducibility of measuring lamina cribrosa pore geometry in human and nonhuman primates with in vivo adaptive optics imaging, Invest Ophthalmol Vis Sci 52(8), 5473-5480 (2011) (hereafter “K. M. Ivers article”). The results obtained using OMAG imaging of the ONH were comparable with those reported in the K. M. Ivers article (average pore area about 1698 μm² with a standard deviation of 1405, and the average elongation ratio 1.72 with a standard deviation of 0.29). Such results demonstrate the usefulness of using OMAG imaging combined with quantitative analysis as an examination tool in future assessment of glaucoma.

An ability to concurrently image and assess microstructure and functional microcirculation of the ONH, as described above for Example 1, opens a new realm of possibilities for diagnosing, monitoring, and therapeutic guidance in the management of disease processes of the eye. The system and method described in Example 1 is also applicable to other tissues, such as the heart, walls of vessels elsewhere in the vascular system, connective tissues such as cartilage and tendon, the central nervous system, and various other internal organs.

The system 200 may be a useful tool for the study of mechanisms associated with physiologic regulation of ONH blood flow, effects of pharmacologic agents and vascular components of pathologic processes associated with ONH disease states. The system 200 may be used for a subject at risk of any ocular pathology, including but not limited to glaucoma, papilledema, idiopathic and inflammatory forms of optic neuritis, and ischemic optic neuropathies.

In some example embodiments, UHS-OMAG and OMAG imaging protocols may be used in combination to achieve 3D data volumes, from which 3D blood flow images may be reconstructed. Additionally, D-OMAG may be used to quantify volumetric, e.g., blood flow properties.

The OMAG imaging protocol comprises performing a repeated scan (i.e., two or more scans at or across the same location) of a sample with a probe beam from a light source, such as the light sources described with reference to FIGS. 1-2. The more scans that are performed for the repeated scan, the more time is required to obtain 3D images. The repeated scan may comprise one or more scanning patterns of the following: a repeated scan at one spatial location (A-scan), a repeated scan at one cross-section (B-scan), and a repeated scan at a tissue volume (C-scan). A repeated scan of the same location is able to capture the data necessary to obtain a microvascular image.

Volumetric properties may include but are not limited to a velocity and a quantity of volume of fluid flow through one or more vessels with summation of volumetric data for the volume, a bulk flow within an ONH, and structural information about a blood supply surrounding and within peripheral regions of the ONH. The peripheral regions of the ONH include, but are not limited to, vessels arising from posterior ciliary arteries, choroidal circulation that enters an optic nerve, and a circle of Zinn-Haller.

Volumetric properties may be used to measure at least one vessel diameter, to quantify a total optic nerve vascular volume, to quantify a vascular volume at each level within an ONH, to measure a prelaminar vascular volume, to measure a total volume of vascular beds of LC, or to measure a flow within a vessel entering the optic nerve.

The volumetric properties may be used to perform an analysis of the waveform of the arterial and/or venous pulse waves within vessels of the optic nerve or retina. Various correlations may also be drawn from such data. For example, correlations may be made between pulse amplitudes of arterial circulation and venous circulation and between time and phase relationships between peaks and troughs of pulse waves of the arterial circulation and the venous circulations. Additionally, pulsatile flow amplitudes of arterial and venous circulation of the optic nerve may be calculated. In some examples, amplitude, phase, and time relationships between pulsatile motions of arterial and venous systems, and fluidics (the use of a fluid to perform operations) of cerebrospinal fluid compartments are determined, including a subarachnoid space surrounding the optic nerve. The fluidics may be determined independently from the amplitude, phase, and time relationships of the pulsatile motions, and the fluidics may then be correlated with the determined amplitude, phase, and time relationships. The fluidics of the cerebrospinal fluid compartment may be determined based on information from the pulsatile behavior, such as the pulsatile motions and pulsatile flow amplitudes.

In one example embodiment, calculating volumetric properties from data obtained comprises determining the volume of functional blood from the volumetric microcirculation image, calculating the physical volume of the scanned tissue to determine a mass of the scanned tissue, and calculating a ratio of volume of functional blood to the mass to determine the volume of blood flow. The volumetric properties may be calculated from the microcirculation image at different tissue depths produced by applying a segmentation algorithm to segment the volumetric images. The volumetric properties may be calculated from a 2D (x-y) projection image produced from a 3D (volumetric, x-y-z) microcirculation image.

In another example embodiment, calculating volumetric properties from data obtained comprises determining the volume of functional blood from the volumetric microcirculation image, calculating the physical volume of the scanned tissue, and calculating the ratio of the volume of functional blood to the physical volume to determine the blood vessel density within the scanned tissue volume. The volumetric properties may be calculated from the microcirculation image at different tissue depths produced by applying a segmentation algorithm to segment the volumetric images. The volumetric properties may be calculated from a 2D (x-y) projection image produced from a 3D (volumetric, x-y-z) microcirculation image.

One exemplary calculation is to calculate retinal blood flow (RBF) from such volumetric data. First, certain retinal arteries or vessels are selected using vessel branches located near the ONH. The axial velocity of each vessel may then be determined from a D-OMAG cross-sectional phase image by calculating the phase difference between adjacent A-lines. The axial velocity V_{Z} may be calculated as:

$${V}_{Z} = \frac{Δϕ \cdot {λ}_{0}}{4 π n Δ {t}_{A}} \qquad Equation 1$$

where λ₀ is the central wavelength, n is the refractive index, Δφ is the phase difference between adjacent A-lines and Δt_{A} is the time interval between adjacent A-lines. A Doppler angle for a vessel and a blood vessel diameter may be determined from 3D vasculature maps.

Absolute blood flow velocity may then be calculated from the axial velocity V_{Z} after the value is corrected by the Doppler angle. The blood flow rate may then be calculated for each vessel by multiplying the absolute velocity with the area of the vessel cross-section.

In one embodiment, the volumetric properties may be calculated for an entire sample. In another embodiment, the volumetric properties may be calculated for a segment or a selected region of a sample. In this embodiment, data for each selected region of the sample is obtained and volumetric properties for each selected region are then independently calculated.

Volumetric properties may be used to correlate a cardiac pulse-induced dynamic movement of lamina cribrosa beams with vascular local and bulk flow measurments within vessels of the ONH and surrounding tissues, to concurrently compare vascular dimensions, surrounding X-Y and 3D connective tissue dimensions, and fluid flow within and surrounding the ONH.

### Example 2

### Imaging and Calculation of Microcirculation Parameters within a Rat Eye

In another example, a system such as system 200 described with reference to FIG. 2 was used to image and analyze the tissue and blood flow in a rat retina, ONH, and surrounding choroid. Quantitative measurements of elevated intraocular pressure (IOP) on vascular beds were determined as well.

Example 2 is discussed in detail in Zhi Z., et al., Impact of Intraocular Pressure on Changes of Blood Flow in the Retina, Choroid, and Optic Nerve Head in Rats Investigated by Optical Microangiography, Biomed Opt. Express 1:3(9):2220-33; 2012. In Example 2, the OMAG system was operated at a wavelength of 1300 nm The axial resolution was 12 μm and the lateral resolution was about 16 μm in air. The maximal imaging speed of the system was 92,000 A-scans per second, and the measured signal to noise ratio (SNR) was about 100 dB at the focus spot of the sampling beam. The total depth range was measured to be about 2.8 mm in air.

At each IOP level, 3D data volumes covering the ONH area were captured using the UHS-OMAG scanning protocol, similar to that described with reference to FIGS. 1 and 2. The raster scanning was performed to capture 256 A-lines within each B-scan and 1000 B-scans for each C-scan. Using a frame rate of 280 frames per second (fps), a single 3D data set was obtained in about 3 seconds. Then, repeated B-scans (using 3000 A-lines per B-scan and a frame rate of 10 fps) at one cross-section for D-OMAG analysis were applied to determine the axial blood flow velocity in selected retinal arteries or veins. A switch between UHS-OMAG and D-OMAG imaging protocols was controlled by software from a computing device, such as the main computing device 222 described with reference to FIG. 2.

3D blood flow images were reconstructed from the 3D data volumes by applying high pass filtering along the slow scan direction to separate the moving blood flow from static tissues. Thereafter calculations such as for an RBF, choroidal, and ONH blood flow discussed above, were performed. In order to assess the effect of IOP on RBF, the flow rates at each level of IOP were normalized and expressed as a percentage of the baseline reading (10 mmHg).

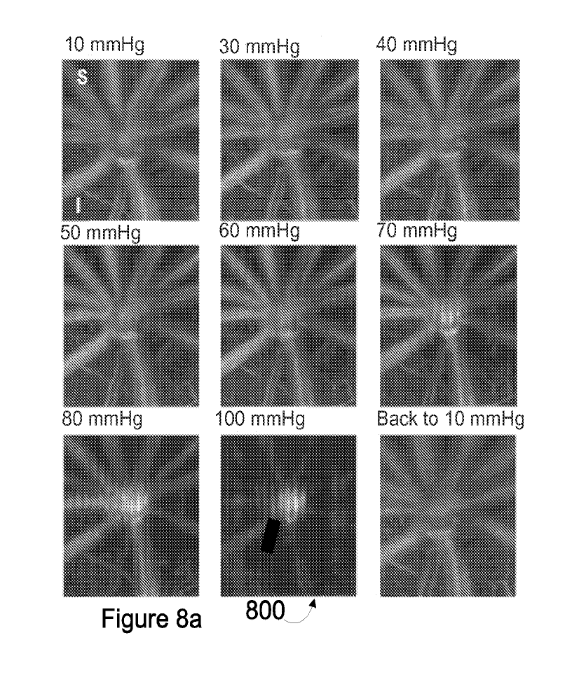

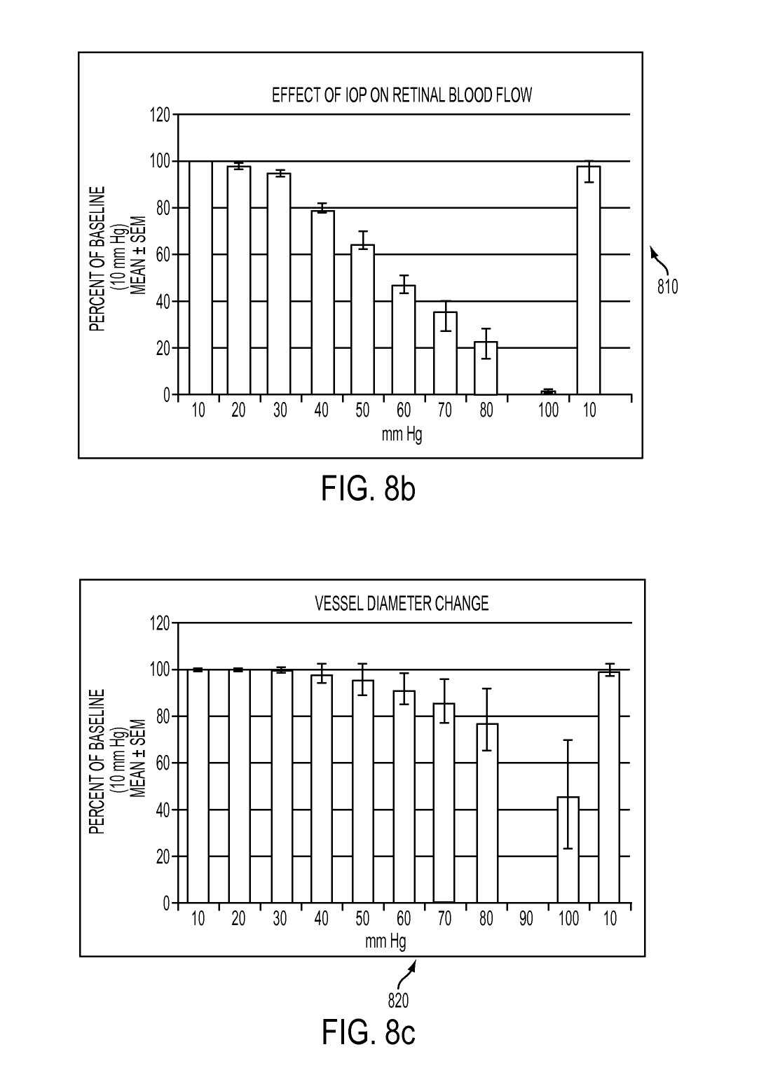

In Example 2, UHS-OMAG microangiogram maps 800, shown in FIG. 8a, of the rat REF showed a progressive decrease in the density of functional capillaries and a decreased diameter of the larger vessels as the IOP was increased from 10 mmHg to 80 mmHg, with near complete obstruction at 100 mmHg.

FIG. 8b depicts a graph 810 the quantification of the effect of elevated IOP on RBF. Average blood flow rate changes from 6 eyes demonstrated an approximately linear decrease in RBF relative to the baseline, starting from 30 mmHg to nearly 0 at 100 mmHg. RBF and flow values reverted to baseline after IOP was returned to 10 mmHg. FIG. 8c depicts a graph 820 illustrating corresponding vessel diameter changes. The changes depicted in FIG. 8c help identify the contribution of velocity and vessel diameter to the reduced blood flow rate, showing that the reduction in vessel diameter was smaller than that of blood flow rate, suggesting that the blood flow rate reduction resulted from decreased flow velocity as well as reduction in vessel diameter.

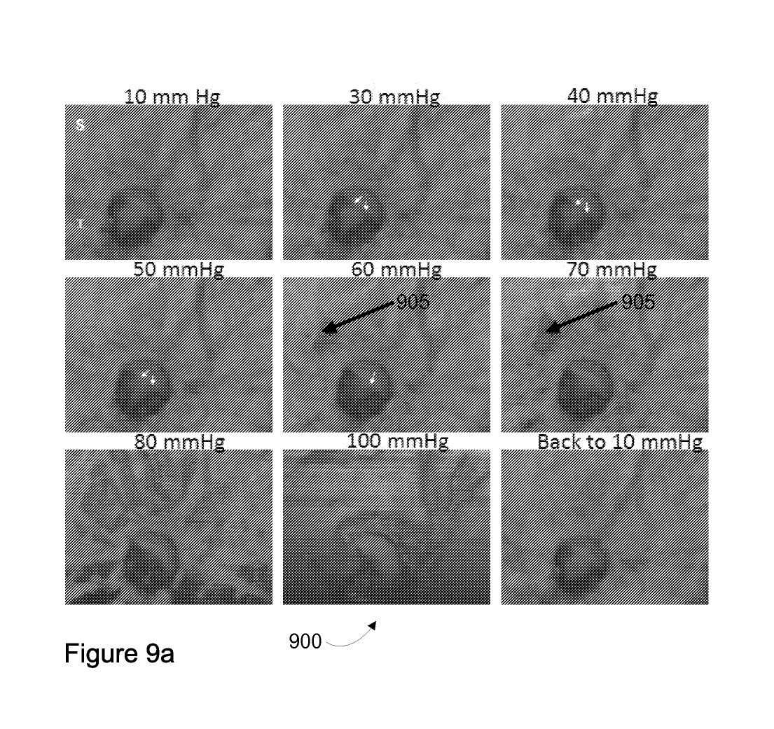

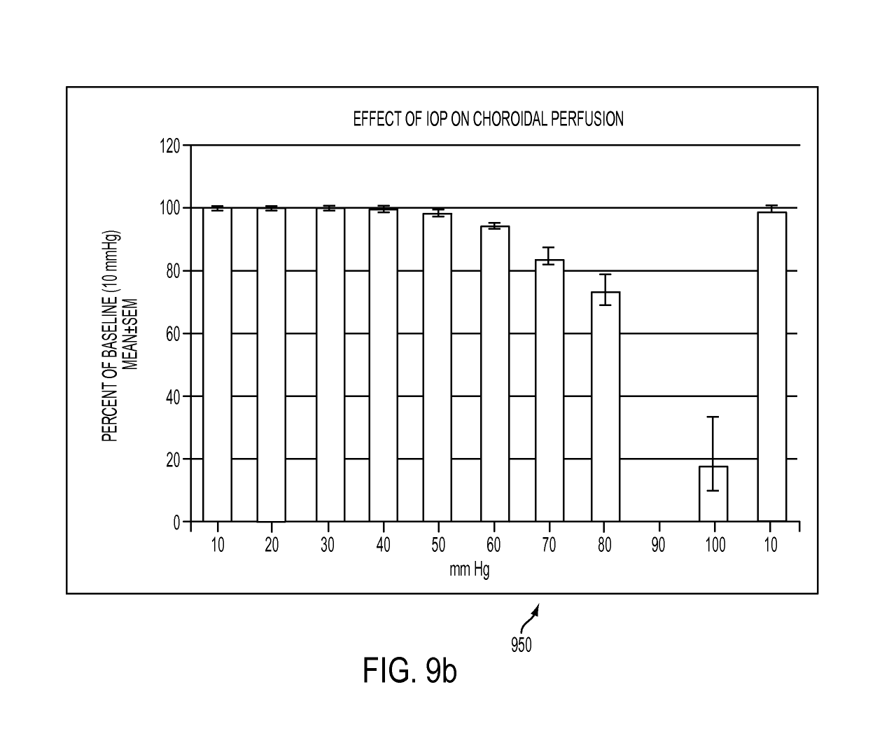

FIG. 9a depicts a UHS-OMAG microangiogram 900 of choroidal and ONH capillary beds at increasing IOP from 10 mmHg to 100 mmHg and back to 10 mmHg, after removal of the retinal vessels, in accordance with at least one embodiment. This revealed, at low IOPs, a dense and intact choroidal capillary bed and choroicapillaris with an opening for the ONH. As the IOP increased, the capillary beds began to show the effects by 60 mmHg, as demonstrated by apparent filling voids 905. All changes reverted to baseline once TOP was returned to 10 mmHg.

To quantitate the reduced choroidal perfusion or filling with increasing IOP, the area of choroidal filling at each TOP level was measured and calculated as a percentage of the choroidal filling seen at baseline (10 mmHg). This is shown in FIG. 9b, which depicts a graph 950 plotting percent of baseline over mmHg.

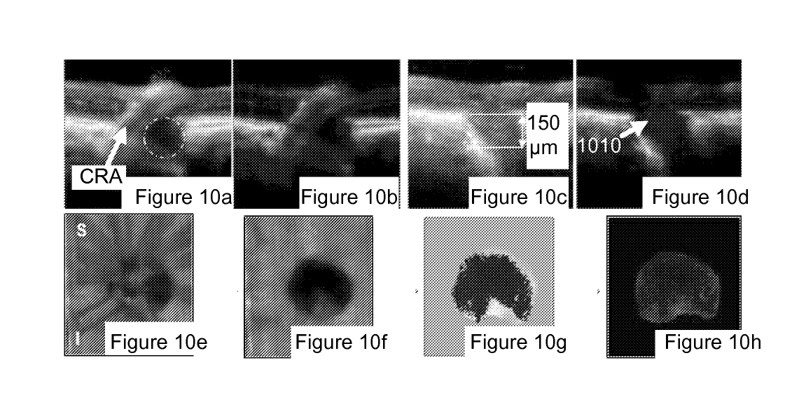

FIGS. 10a-d depict OCT structural images and corresponding flow images across the ONH region at two TOP levels (20 mmHg and 60 mmHg, respectively). Capillary flow signals within the ONH are visualized in FIG. 10d with arrow 1010, however, overlying retinal vessels may shadow the structures within the ONH which results in low signal strength in the dashed circle of the OCT image of FIG. 10a, causing some capillary perfusion to be undetectable in FIG. 10b.

FIGS. 10e-h depict steps in quantitation of ONH blood perfusion. These steps include segmentation of the retinal vasculature from anterior view 3D vasculature maps of FIGS. 10e-f, identifying the blood flow signal pixel map at FIG. 10g, and the ONH volume used for the percentage of perfusion calculation of FIG. 101-h.

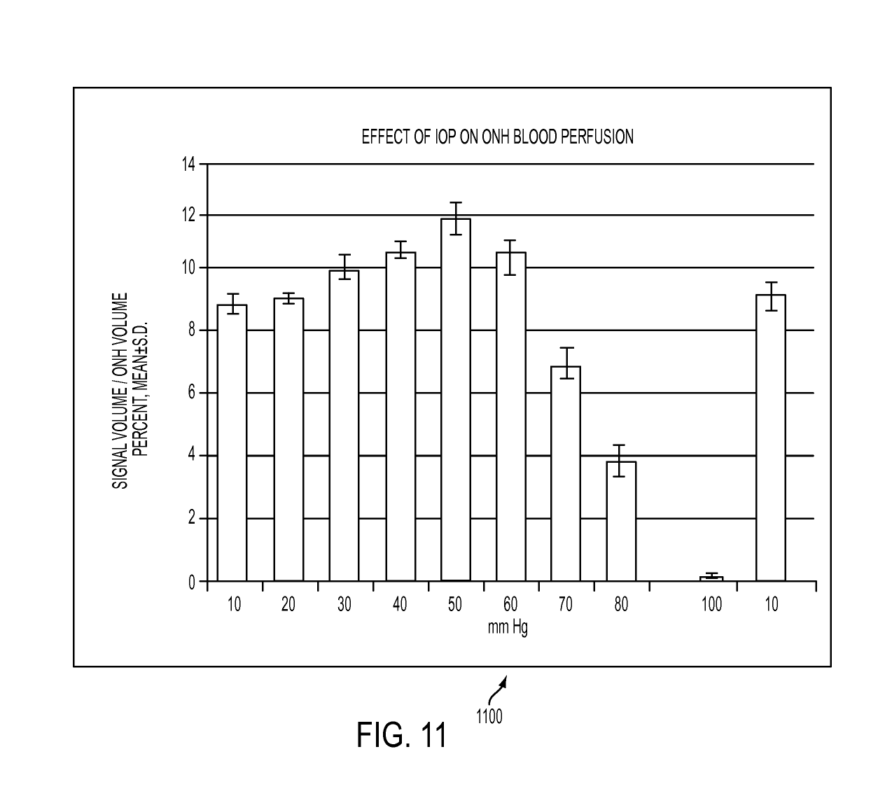

FIG. 11 depicts a graph 1100 showing the effect of IOP on ONH blood perfusion as a signal volume percent plotted over mmHg. The graph 1100 shows an increase in perfusion at lower IOPs. ONH perfusion reverted to baseline when IOP returned to 10 mmHg.

Elevation of IOP is known to affect retinal perfusion, and may play a role in the development of optic nerve damage in some glaucoma patients. However, unless perfusion of the back of the eye can be determined, it is difficult to be certain what role reduced perfusion may play in the effects of elevated IOP on the relevant tissues. For these reasons, developing non-invasive methods of imaging and measuring blood flow in the retina, choroid and ONH is important for both clinical management and experimental research in glaucoma.

The above described systems and methods may be used for a number of optic nerve assessments, including but not limited to: tomographic measurement of the structural organization of the optic nerve, tomographic measurements permitting quantitation of the size and shape of pores in each of the laminar beams, quantitation of the total pore size within the layers of the laminar beams, quantitative assessment of changes in size and configuration of the laminar pores, assessment of prelaminar vascular volume, assessment of total volume of vascular beds of LC, quantification of total or global optic nerve vascular volume, quantification of vascular volume at least level within the ONH, characterization of flow patterns of the microcirculation within each level of the prelaminar and laminar portion of the optic nerve, characterization of vascular flow patterns within the vessels that arise from the posterior ciliary arteries and pass through the sclera rim, characterization of the region of the circle of Zinn-Haller, characterization of flow patterns of vessels entering the optic nerves from the choroidal circulation, measurement of velocity of flow through vessels, measurement of vessel diameters by means of both structural and phase-based techniques, quantitative assessment of flow within individual vessels entering the optic nerve, assessment of the effect of medications on the above vascular parameters, and assessments of IOP effects on the vascular parameters.

The determination of microvascular functions may be used to diagnose, provide a prognosis, monitor treatment and guide treatment decisions for a disorder of the sample of a subject. The treatment may include medical, laser, or surgical intervention. A treatment decision may be based on the prognosis, monitoring or assessment of current properties of the tissues or regions of the tissue conducted in accordance with the determination of microvascular functions performed in the manner described above.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims, along with the full scope of equivalents to which such claims are entitled. It is also to be understood that the terminology used herein is for the purpose of describing particular embodiments only, and is not intended to be limiting.

What is claimed is:

1. A method for determining microvascular functions in a sample of a subject comprising:
performing a repeated scan of the sample with a probe beam from a light source, wherein the repeated scan comprises two or more scans at the same location;
obtaining one or more spectral interference signals from the sample during the scan;
extracting data from the spectral interference signals concerning cell, tissue, or particle motion within the sample; and
calculating volumetric properties from the data indicative of fluid motion within the sample.

2. The method of claim 1, wherein performing the repeated scan comprises a D-OMAG imaging protocol and performing the plurality of fast scans on the fast scan axis and the plurality of slow scans on the slow scan axis comprises a UHS-OMAG imaging protocol, and a physical computer-readable storage medium executes instructions to switch between running the D-OMAG scanning protocol and the UHS-OMAG imaging protocol.

3. The method of claim 1, wherein the repeated scan comprises one or more scanning patterns selected from the group consisting of a repeated scan at one spatial location, a repeated scan at one cross-section, and a repeated scan at a tissue volume.

4. The method of claim 1, wherein calculating the volumetric properties from the data further comprises:
segmenting selected regions of the sample and obtaining data for each region.

5. The method of claim 1, wherein calculating the volumetric properties from the data further comprises:
determining a volume of functional blood from a volumetric microcirculation image;
calculating a physical volume of the sample to determine a mass of the sample; and
calculating a ratio of volume of functional blood to the mass to determine the volume of blood flow.

6. The method of claim l, wherein calculating the volumetric properties from the data further comprises:
determining a volume of functional blood from a volumetric microcirculation image;
calculating a physical volume of the sample; and
calculating a ratio of the volume of functional blood to the physical volume to determine a blood vessel density within the sample.

7. The methods of claim 5, wherein the volumetric properties from the data are calculated from the microcirculation image at different tissue depths produced by applying a segmentation algorithm.

8. The methods of claim 5, wherein the volumetric properties from the data are calculated from a 2D projection image produced from a 3D microcirculation image.

9. The method of claim 1, wherein calculating the volumetric properties from the data comprises:
determining an axial velocity for the one or more vessels from a phase difference between adjacent A-lines captured from the scanning;
determining a Doppler angle and a diameter of the one or more vessels from vasculature maps captured from the repeated scans;
correcting the axial velocity using the Doppler angle;
calculating an approximate absolute velocity from the corrected axial velocity;
calculating an area of a cross-section of the one or more vessels from the diameter; and
multiplying the absolute velocity with the area of the vessel cross-section to obtain a blood flow rate for the one or more vessels.

10. The method of claim 1, wherein the volumetric properties include one or more of a velocity, a quantity or volume of fluid flow through one or more vessels with summation of volumetric data for the volume, a bulk flow within an optic nerve head (ONH), and structural information about a blood supply surrounding and within peripheral regions of the ONH.

11. The method of claim 10, wherein the peripheral regions of the ONH include vessels arising from posterior ciliary arteries, choroidal circulation that enters an optic nerve, and a circle of Zinn-Haller.

12. The method of claim 10, further comprising one or more of:
correlating a cardiac pulse-induced dynamic movement of lamina cribrosa beams with vascular local and bulk flow measurements within vessels of the ONH and surrounding tissues;
correlating pulse amplitudes of arterial circulation and venous circulation; and
correlating time and phase relationships between peaks and troughs of pulse waves of the arterial circulation and the venous circulations.

13. The method of claim 10, further comprising:
calculating pulsatile flow amplitudes of arterial and venous circulation of the optic nerve.

14. The method of claim 10, further comprising:
determining amplitude, phase, and time relationships between pulsatile motions of arterial and venous systems.

15. The method of claim 14, further comprising:
determining fluidics of a cerebrospinal fluid compartment based on the pulsatile motions.

16. The method of claim 10, further comprising:
concurrently comparing vascular dimensions, surrounding X-Y and 3D connective tissue dimensions, and fluid flow within and surrounding the ONH.

17. The method of claim 1, wherein the subject is at risk of an ocular pathology or has an ocular pathology and wherein the ocular pathology is one or more of glaucoma, papilledema, inflammatory neuropathies, and ischemic neuropathies.

18. The method of claim 1, wherein the method is used to measure at least one vessel diameter, to quantify a total optic nerve vascular volume, to quantify a vascular volume at each level within an ONH, to measure a prelaminar vascular volume, to measure a total volume of vascular beds of LC, or to measure a flow within a vessel entering the optic nerve.

19. The method of claim 1, wherein the method is used to diagnose, provide a prognosis, monitor treatment, or provide guidance in medical, laser or surgical management for a disorder of the tissue of the skin, heart, vasculature microcirculation, connective tissue structures, internal organs, or central nervous system structures.

20. A system for measuring microcirculation comprising:
an optical coherence tomography probe;
an optical circulator;
a coupler;
a spectrometer; and
a physical computer-readable storage medium;
wherein the system is configured to acquire images from living tissue;
wherein the physical computer-readable storage medium has stored thereon instructions executable by a device to cause the device to perform functions to extract microcirculation data from images acquired from optical coherence tomography scans of the tissue, the functions comprising:
determining a phase difference and a time interval between adjacent A-lines from the acquired images;
calculating an axial velocity for the at least one vessel from the determined phase difference and the time interval;
determining a Doppler angle and a diameter of at least one vessel from the acquired images; and
calculating blood flow velocity from the axial velocity, the Doppler angle, and the diameter of the at least one vessel.
